(19) United States
(12) Patent Application Publication
(10) Pub. No.: US 2024/0252409 A1
(43) Pub. Date: Aug. 1, 2024

(54) RESHAPING COMPOSITION FOR KERATIN FIBERS

(71) Applicant: KAO GERMANY GMBH, Darmstadt (DE)

(72) Inventors: Steven BREAKSPEAR, Darmstadt (DE); Bernd NÖCKER, Darmstadt (DE); Peter BAUER, Darmstadt (DE); Susann WARMBIER, Darmstadt (DE)

(73) Assignee: KAO GERMANY GMBH, Darmstadt (DE)

(21) Appl. No.: 18/289,668

(22) PCT Filed: May 13, 2022
(86) PCT No.: PCT/EP2022/063070

§ 371 Date: Nov. 6, 2023

(30) Foreign Application Priority Data
May 18, 2021 (EP) 21174366.1

(51) Int. Cl. A61K 8/365; A61K 8/34; A61Q 5/04

(52) U.S. Cl. CPC A61K 8/365; A61K 8/345; A61Q 5/04; A61K 2800/88

(57) ABSTRACT

Aqueous straightening compositions for keratin fibers, two-chamber packaging devices, kits-of-parts, and straightening processes are provided. Said compositions have a pH in the range of 1 to 6 and comprise a) one or more tridentate carboxylic acid, and/or their salt(s), and/or their hydrates, and/or their mixtures, and b) one or more compound(s) C2-C4 trialcohol(s), and/or their mixtures, wherein the total concentration of compound(s) according to group a) is 1% by weight or more, calculated to the total weight of the composition.

### FIELD OF THE INVENTION

The invention is directed to the provision of a straightening composition for keratin fibers, a straightening process, and a kit-of-parts for straightening.

### BACKGROUND OF THE INVENTION

Straightening treatments for keratin fibers based on formaldehyde, strong alkali compositions (lanthionization) or reducing/oxidizing processes are highly effective, but these processes usually cause significant hair damage. In the past decade, treatments based on glyoxylic acid became more popular, but for some clients the straightening effect is too permanent or too strong.
WO2015/075062 discloses hair-straightening compositions comprising citric acid.
WO2013/174690 discloses hair-straightening compositions comprising citric acid and gluconolactone. The hair treated with heat.
WO2017/137182 discloses hair-straightening compositions comprising citric acid and silk sericin.
WO2013/174703 discloses hair-straightening compositions comprising citric acid and sugar alcohols. The hair treated with heat.

### SUMMARY OF THE INVENTION

The first object of the present invention is an aqueous straightening composition for keratin fibers, preferably for human keratin fibers, more preferably for human hair, having a pH in the range of 1 to 6 and comprising:
a) one or more bidentate or tridentate carboxylic acid, and/or their salt(s), and/or their hydrates, and/or their mixtures,
b) one or more C₂-C₄ dialcohol(s) or trialcohol(s), and/or their mixtures, wherein the total concentration of compound(s) according to group a) is 1% by weight or more, calculated to the total weight of the composition.

The second object of the present invention is a two-chamber packaging device having:
a first chamber comprising a first aqueous composition comprising one or more compound(s) according to group a) as defined in above, and
a second chamber comprising a second aqueous composition comprising one or more compound(s) according to group b) as defined above.

The third object of the present invention is a kit-of-parts having a first component with an aqueous composition comprising one or more compound(s) according to group a) as defined above, and a second component with an aqueous composition comprising one or more compound(s) according to group b) as defined above.

The fourth object of the present invention is a straightening process for keratin fibers comprising the steps in the following order of:
i) providing the composition as defined above for the straightening composition, or providing and mixing the first and second component as defined above for the kit, or providing and dispensing and optionally mixing the compositions as defined above for the two-chamber packaging, yielding a composition having a pH in the range of 1 to 6, preferably in the range of 1.25 to 5, more preferably in the range of 1.5 to 4.5, further more preferably in the range of 2 to 4,
ii) applying the composition of step i) onto keratin fibers,
iii) optionally covering the keratin fibers with a moisture barrier,
iv) leaving the composition onto the keratin fibers for a time period of 1 min to 24 h, preferably for a time period of 1 min to 120 min,
v) optionally rinsing-off the hair,
vi) drying the hair,
vii) applying heat to the keratin fibers at a temperature in the range of 50° C. to 230° C.,
viii) rinsing-off the keratin fibers and optionally drying the keratin fibers.

### DETAILED DESCRIPTION OF THE INVENTION

Inventors of the present invention have unexpectedly found out that a composition according to claim 1 effectively straightens hair. The straightening effect lasted over at least 10 hair washes. Furthermore, the hair was less damaged in comparison to other known straightening techniques.

### Aqueous Straightening Composition

The present invention is directed to an aqueous straightening composition for keratin fibers, preferably for human keratin fibers, more preferably for human hair, having a pH in the range of 1 to 6 and comprising:
a) one or more bidentate or tridentate carboxylic acid, and/or their salt(s), and/or their hydrates, and/or their mixtures,
b) one or more C₂-C₄ dialcohol(s) or trialcohol(s), and/or their mixtures, wherein the total concentration of compound(s) according to group a) is 1% by weight or more, calculated to the total weight of the composition.

The term ‘aqueous’ within the meaning of the present invention requires the composition to have a water content of 50% by weight or more, preferably to have a water content of 60% by weight or more, more preferably to have a water content of 70% by weight or more, calculated to the total weight of the composition.

It is preferred from the viewpoint of straightening efficiency that the pH of the composition is 5 or lower, preferably the pH is 4.5 or lower, more preferably the pH is 4 or lower.

It is preferred from the viewpoint of cosmetic safety that the pH of the composition is 1.25 or higher, preferably 1.5 or higher, further more preferably 2 or higher.

For attaining the above-mentioned effects, it is preferred that the pH of the composition is in the range of 1.25 to 5, more preferably in the range of 1.5 to 4.5, further more preferably in the range of 2 to 4.

It is preferred from the viewpoint of minimizing damage to keratin fibers that the composition comprises less than 1% by weight of reducing agent(s), preferably less than 0.1% by weight of reducing agent(s), calculated to the total weight of the composition.

It is further preferred from the viewpoint of minimizing damage to keratin fibers that the composition is free of reducing agents.

It is preferred from the viewpoint of minimizing damage to keratin fibers that the composition comprises less than 1% by weight of oxidizing agent(s), preferably less than 1% by weight of oxidizing agent(s), calculated to the total weight of the composition.

It is preferred from the viewpoint of minimizing damage to keratin fibers that the composition is free of oxidizing agents.

It is further preferred from the viewpoint of cosmetic applicability that the composition of the present invention is a temporary straightening composition for keratin fibers, preferably a temporary straightening composition for keratin fibers with heat at 50° C. or above, more preferably with heat at 120° C. or above.

It is further preferred from the viewpoint of cosmetic applicability that the composition of the present invention is a temporary straightening composition.

### Compound(s) According to Group a)

The composition of the present invention comprises one or more bidentate or tridentate carboxylic acid, and/or their salt(s), and/or their mixtures, as compound(s) according to group a).

Suitable compound(s) according to group a) is/are bidentate carboxylic acid(s), preferably it is oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures.

The more preferred compound(s) according to group a) from the viewpoint of cosmetic acceptance is/are selected from oxalic acid, malonic acid, succinic acid, maleic acid, fumaric acid, and/or their salt(s), and/or their hydrates, and/or their mixtures.

The most preferred compound(s) according to group a) from the viewpoint of cosmetic acceptance is/are according to group a) is/are succinic acid, and/or its salt(s), and/or its hydrate(s).

Further suitable compound(s) according to group a) is/are tridentate carboxylic acid(s), preferably it is/are citric acid, isocitric acid, aconitic acid, propane-1,2,3-tricarboxylic acid, trimesic acid, and/or their salt(s), and/or their hydrates, and/or their mixtures.

More preferably one or more compound(s) according to group a) is/are selected from citric acid, isocitric acid, aconitic acid, propane-1,2,3-tricarboxylic acid, trimesic acid, and/or their salt(s), and/or their hydrates, and/or their mixtures, form the viewpoint of straightening efficiency.

The more preferred compound(s) according to group a) from the viewpoint of straightening efficiency is/are selected from citric acid or isocitric acid, and/or their salt(s), and/or their hydrates, and/or their mixtures.

The most preferred compound(s) according to group a) is citric acid, and/or its salt(s), and/or its hydrates.

Therefore, the present disclosure is also directed to an aqueous straightening composition for keratin fibers, preferably for human keratin fibers, more preferably for human hair, having a pH in the range of 1 to 6 and comprising:
a) one or more tridentate carboxylic acid, and/or their salt(s), and/or their hydrates, and/or their mixtures, preferably it is/are selected from citric acid or isocitric acid, and/or their salt(s), and/or their hydrates, and/or their mixtures, more preferably it is/are selected from citric acid or isocitric acid, and/or their salt(s), and/or their hydrates, and/or their mixtures, still more preferably it is citric acid, and/or its salt(s), and/or its hydrates and/or one or more bidentate carboxylic acid(s), preferably it is oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures,
b) one or more C₂-C₄ dialcohol(s) or trialcohol(s), and/or their mixtures, wherein the total concentration of compound(s) according to group a) is 1% by weight or more, calculated to the total weight of the composition.

It is preferred from the viewpoint of straightening efficiency that the total concentration of compound(s) according to group a) is 1% by weight or more, more preferably it is 2% by weight or more, further more preferably it is 3% by weight or more, calculated to the total weight of the composition.

It is further preferred from the viewpoint of cosmetic safety that the total concentration of compound(s) according to group a) is 30% by weight or less, preferably 20% by weight or less, more preferably 15% by weight or less, still further more preferably 10% by weight or less, still further more preferably 8% by weight or less.

For attaining the above-mentioned effects, it is preferred that the total concentration of compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of bidentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of succinic acid and/or its salt(s) and/or its hydrates as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of tridentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of citric acid and/or its salt(s) and/or its hydrates as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

### Compound(s) According to Group b)

The composition of the present invention comprises one or more C₂-C₄ dialcohol(s) or trialcohol(s), and/or their mixtures, as compound(s) according to group b).

Suitable compound(s) according to group b) is/are selected from ethylene glycol, propylene glycol, butylene glycol, glycerol, and/or their mixtures. The preferred compound(s) according to group b) is glycerol, from the viewpoint of straightening efficiency.

It is preferred from the viewpoint of straightening efficiency that the weight ratio of compound(s) according to a) to compound(s) according to b) is in the range of 0.2 to 5, preferably it is in the range of 0.25 to 3, more preferably it is in the range of 0.5 to 2, further more preferably it is in the range of 0.5 to 1, or is in the range of 0.25 to 3, more preferably it is in the range of 0.5 to 3, further more preferably it is in the range of 1 to 3.

It is further preferred that total concentration of one or more compound(s) according to group b) is in the range of 0.5% to 15% by weight, preferably in the range of 1% to 10% by weight, further more preferably in the range of 2% to 8% by weight, calculated to the total weight of the composition.

It is preferred from the viewpoint of straightening efficiency that the total concentration of one or more compound(s) according to group b) is 0.5% by weight or more, preferably it is 1% by weight or more, further more preferably it is 2% by weight or more, calculated to the total weight of the composition.

It is preferred from the viewpoint of cosmetic safety that the total concentration of one or more compound(s) according to group b) is 15% by weight or less, preferably it is 10% by weight or less, further more preferably it is 8% by weight or less, calculated to the total weight of the composition.

For attaining the above-mentioned effects, it is preferred that the total concentration of one or more compound(s) according to group b) is in the range of 0.5% to 15% by weight, preferably in the range of 1% to 10% by weight, further more preferably in the range of 2% to 8% by weight, calculated to the total weight of the composition.

The disclosure of the present invention is also directed to an aqueous straightening composition for keratin fibers, preferably for human keratin fibers, more preferably for human hair, having a pH in the range of 1 to 6 and comprising:
a) citric acid and/or its salt(s), and/or its hydrates,
b) glycerol,
wherein the total concentration of compound(s) according to group a) is 1% by weight or more, preferably it is in the range of 1% to 30% by weight, more preferably in the range of 2% to 20% by weight, further more preferably in the range of 3% to 15% by weight, still further more preferably in the range of 3% to 10% by weight, still further more preferably in the range of 3% to 8% by weight, calculated to the total weight of the composition.

The disclosure of the present invention is also directed to an aqueous reshaping composition for keratin fibers, preferably for human keratin fibers, more preferably for human hair, having a pH in the range of 1 to 6 and comprising:
a) citric acid and/or its salt(s), and/or its hydrates,
b) glycerol,
wherein the total concentration of compound(s) according to group a) is 1% by weight or more, preferably it is in the range of 1% to 30% by weight, more preferably in the range of 2% to 20% by weight, further more preferably in the range of 3% to 15% by weight, still further more preferably in the range of 3% to 10% by weight, still further more preferably in the range of 3% to 8% by weight, calculated to the total weight of the composition, and
wherein the weight ratio of compound(s) according to a) to compound(s) according to b) is in the range of 0.2 to 5.

### Compound(s) According to Group c)

It is preferred from the viewpoint of conditioning that it comprises one or more conditioning compound(s) as compound(s) according to group c), preferably selected from silicones, fatty alcohols, and/or cationic surfactants, and/or their salt(s), and/or their mixtures.

Suitably, one or more compound(s) according to group c) is/are silicones, preferably aminated silicones.

It is also preferred that one or more compound(s) according to group c) is/are cationic surfactant(s), and/or their salt(s), preferably selected from quaternary ammonium surfactants and/or amidopropylamino surfactants, and/or their salt(s), and/or their mixtures.

Suitable compounds are cetrimonium chloride, behentrinonium chloride, behenamidopropyl dimethylamine, and/or their salt(s), and/or their mixtures.

It is preferred from the viewpoint of conditioning effect that the total concentration of one or more compound(s) according to group c) is in the range of 0.1% to 5% by weight, preferably in the range of 0.25% to 3% by weight, more preferably in the range of 0.5% to 2% by weight, calculated to the total weight of the composition.

In one aspect of the present invention that the total concentration of silicones, preferably aminated silicones, as one or more compound(s) according to group c) is in the range of 0.1% to 5% by weight, preferably in the range of 0.25% to 3% by weight, more preferably in the range of 0.5% to 2% by weight, calculated to the total weight of the composition.

In one aspect of the present invention that the total concentration of cationic surfactant(s), and/or their salt(s), preferably selected from quaternary ammonium surfactants and/or amidopropylamino surfactants, and/or their salt(s), and/or their mixtures, as one or more compound(s) according to group c) is in the range of 0.1% to 5% by weight, preferably in the range of 0.25% to 3% by weight, more preferably in the range of 0.5% to 2% by weight, calculated to the total weight of the composition.

### Two-Chamber Packaging

The present invention is also directed to a two-chamber packaging device having:
a first chamber comprising a first aqueous composition comprising one or more compound(s) according to group a) as defined above, and
a second chamber comprising a second aqueous composition comprising one or more compound(s) according to group b) as defined above.

The first and second chamber compositions are kept separate until directly prior to use onto keratin fibers. The mixture of the compositions has a pH in the range of 1 to 6, preferably in the range of 1.25 to 5, more preferably in the range of 1.5 to 4.5, further more preferably in the range of 2 to 4.

From this viewpoint, it is preferred that the first and second chamber compositions have a pH in the range of 1 to 6, preferably in the range of 1.25 to 5, more preferably in the range of 1.5 to 4.5, further more preferably in the range of 2 to 4.

In another aspect, the first chamber composition has a pH in the range of 1 to 6, preferably in the range of 1.25 to 5, more preferably in the range of 1.5 to 4.5, further more preferably in the range of 2 to 4, and the second chamber composition has a pH in the range of 7 to 12. However, the mixtures of the compositions obtained directly prior to application onto keratin fibers has a pH in the range of 1 to 6, preferably in the range of 1.25 to 5, more preferably in the range of 1.5 to 4.5, further more preferably in the range of 2 to 4.

In another aspect, the first chamber composition has a pH in the range of 7 to 12, and the second chamber composition has a pH in the range of 1 to 6, preferably in the range of 1.25 to 5, more preferably in the range of 1.5 to 4.5, further more preferably in the range of 2 to 4. However, the mixtures of the compositions obtained directly prior to application onto keratin fibers has a pH in the range of 1 to 6, preferably in the range of 1.25 to 5, more preferably in the range of 1.5 to 4.5, further more preferably in the range of 2 to 4.

In one aspect of the present invention, the packaging container comprises a valve configured to subsequently or simultaneously dispense from two chambers upon actuation.

The packaging container may be made of any suitable material, for example any suitable metal, single constituent or composite plastics.

### Kit-of-Parts

The present invention is also directed to a kit-of-parts having a first component with an aqueous composition comprising one or more compound(s) according to group a) as defined above, and
a second component with an aqueous composition comprising one or more compound(s) according to group b) as defined above.

The first and second components are kept separate until directly prior to use onto keratin fibers. The mixture of the components has a pH in the range of 1 to 6, preferably in the range of 1.25 to 5, more preferably in the range of 1.5 to 4.5, further more preferably in the range of 2 to 4.

From this viewpoint, it is preferred that the first and second components of the kit have a pH in the range of 1 to 6, preferably in the range of 1.25 to 5, more preferably in the range of 1.5 to 4.5, further more preferably in the range of 2 to 4.

In another aspect, the first component of the kit has a pH in the range of 1 to 6, preferably in the range of 1.25 to 5, more preferably in the range of 1.5 to 4.5, further more preferably in the range of 2 to 4, and the second component of the kit has a pH in the range of 7 to 12. However, the mixtures of the components obtained directly prior to application onto keratin fibers has a pH in the range of 1 to 6, preferably in the range of 1.25 to 5, more preferably in the range of 1.5 to 4.5, further more preferably in the range of 2 to 4.

In another aspect, the first component of the kit has a pH in the range of 7 to 12, and the second component has a pH in the range of 1 to 6, preferably in the range of 1.25 to 5, more preferably in the range of 1.5 to 4.5, further more preferably in the range of 2 to 4. However, the mixtures of the components obtained directly prior to application onto keratin fibers has a pH in the range of 1 to 6, preferably in the range of 1.25 to 5, more preferably in the range of 1.5 to 4.5, further more preferably in the range of 2 to 4.

It is preferred from the viewpoint of conditioning that the first component and/or the second component further comprise one or more compound(s) according to group c) as defined above. The concentration ranges for the compound(s) according to group c) as defined above are applicable to each of the individual components of the kit.

It is preferred from the viewpoint of minimizing damage to keratin fibers that the first and second component comprise less than 1% by weight of reducing agent(s), preferably less than 0.1% by weight of reducing agent(s), calculated to the total weight of the composition, more preferably they are free of reducing agents.

It is further preferred from the viewpoint of minimizing damage to keratin fibers that the first and second component comprise less than 1% by weight of oxidizing agent(s), preferably less than 1% by weight of oxidizing agent(s), calculated to the total weight of the composition, more preferably they are free of oxidizing agents.

### Straightening Process

The present invention is also directed to a straightening process for keratin fibers comprising the steps in the following order of:
i) providing the composition as defined above for the straightening composition, or providing and mixing the first and second component as defined above for the kit, or providing and dispensing and optionally mixing the compositions as defined above for the two-chamber packaging, yielding a composition having a pH in the range of 1 to 6, preferably in the range of 1.25 to 5, more preferably in the range of 1.5 to 4.5, further more preferably in the range of 2 to 4,
ii) applying the composition of step i) onto keratin fibers,
iii) optionally covering the keratin fibers with a moisture barrier,
iv) leaving the composition onto the keratin fibers for a time period of 1 min to 24 h, preferably for a time period of 1 min to 120 min,
v) optionally rinsing-off the hair,
vi) drying the hair,
vii) applying heat to the keratin fibers at a temperature in the range of 50° C. to 230° C.,
viii) rinsing-off the keratin fibers and optionally drying the keratin fibers.

Preferably, the compositions of step ii) are applied onto dry keratin fibers.

It is preferred from the viewpoint of cosmetic applicability that the moisture barrier of step iii) is aluminium foil or a plastic wrapping.

It is preferred from the viewpoint of straightening performance that the temperature of step vii) is in the range of 90° C. to 230° C., preferably in the range of 120° C. to 230° C., more preferably in the range of 150° ° C. to 230° C.

It is preferred from the viewpoint of cosmetic applicability that it is a process for temporary straightening keratin fibers, preferably for temporary straightening keratin fibers.

It is preferred from the viewpoint of straightening performance that the time period of step iii) is in the range of 2 min to 60 min, preferably in the range of 5 min to 45 min, more preferably in the range of 10 min to 40 min.

It is preferred from the viewpoint of cosmetic applicability that heat in step vii) is applied with a straightening iron.

The disclosure of the present invention is also directed to:

<1> An aqueous straightening composition for keratin fibers, preferably for human keratin fibers, more preferably for human hair, having a pH in the range of 1 to 6 and comprising:
a) one or more bidentate or tridentate carboxylic acid, and/or their salt(s), and/or their mixtures,
b) one or more C₂-C₄ dialcohol(s) or trialcohol(s), and/or their mixtures, wherein the total concentration of compound(s) according to group a) is 1% by weight or more, calculated to the total weight of the composition.

<2> The composition of clause <1>, characterized in that one or more compound(s) according to group a) is/are a bidentate carboxylic acid(s) and/or its/their salt(s), and/or its/their hydrates, preferably it is oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures.

<3> The composition according to any of the clauses <1> and/or <2> characterized in that one or more compound(s) according to group a) is/are selected from oxalic acid, malonic acid, succinic acid, maleic acid, fumaric acid, and/or their salt(s), and/or their hydrates, and/or their mixtures.

<4> The composition according to any of the clauses <1> to <3> characterized in that one or more compound(s) according to group a) is/are succinic acid, and/or its salt(s), and/or its hydrate(s).

<5> The composition according to any of the clauses <1> to <4> characterized in that one or more compound(s) according to group a) is/are a tridentate carboxylic acid, and/or its/their salt(s), and/or its/their hydrates.

<6> The composition according to any of the clauses <1> and/or <5> characterized in that one or more tridentate carboxylic acid(s) as compound(s) according to group a) is/are citric acid, isocitric acid, aconitic acid, propane-1,2,3-tricarboxylic acid, trimesic acid, and/or their salt(s), and/or their hydrates, and/or their mixtures.

<7> The composition according to any of the clauses <1> to <6> characterized in that one or more compound(s) according to group a) is/are citric acid, isocitric acid, aconitic acid, propane-1,2,3-tricarboxylic acid, trimesic acid, and/or their salt(s), and/or their hydrates, and/or their mixtures.

<8> The composition according to any of the clauses <1> to <7> characterized in that one or more compound(s) according to group a) is/are selected from citric acid or isocitric acid, and/or their salt(s), and/or their hydrates, and/or their mixtures.

<9> The composition according to any of the clauses <1> to <8> characterized in that one or more compound(s) according to group a) is citric acid, and/or its salt(s), and/or its hydrates.

<10> The composition according to clause <1> characterized in that one or more compound(s) according to group a) is/are a bidentate carboxylic acid(s) and/or its/their salt(s), and/or its/their hydrates, preferably it is oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures, or one or more tridentate carboxylic acid(s), preferably citric acid, isocitric acid, aconitic acid, propane-1,2,3-tricarboxylic acid, trimesic acid, and/or their salt(s), and/or their hydrates, and/or their mixtures.

<11> The composition according to any of the clauses <1> to <10> characterized in that one or more compound(s) according to group a) is/are is/are succinic acid, and/or its salt(s), and/or its hydrate(s), citric acid, and/or its salt(s), and/or its hydrates, and/or their mixtures.

<12> The composition according to any of the clauses <1> to <11> characterized in that the total concentration of compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<13> The composition according to any of the clauses <1> to <12> characterized in that the total concentration of bidentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<14> The composition according to any of the clauses <1> to <13> characterized in that the total concentration of succinic acid and/or its salt(s) and/or its hydrates as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<15> The composition according to any of the clauses <1> to <14> characterized in that the total concentration of tridentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<16> The composition according to any of the clauses <1> to <15> characterized in that the total concentration of citric acid and/or its salt(s), and/or its hydrates, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<17> The composition according to any of the clauses <1> to 16> characterized in that one or more compound(s) according to group b) is/are selected from ethylene glycol, propylene glycol, butylene glycol, glycerol, and/or their mixtures.

<18> The composition according to any of the clauses <1> to <17> characterized in that one or more compound(s) according to group b) is glycerol.

<19> The composition according to any of the clauses <1> to <18> characterized in that the weight ratio of compound(s) according to a) to compound(s) according to b) is in the range of 0.2 to 5, preferably it is in the range of 0.25 to 3, more preferably it is in the range of 0.5 to 2, further more preferably it is in the range of 0.5 to 1, or is in the range of 0.25 to 3, more preferably it is in the range of 0.5 to 3, further more preferably it is in the range of 1 to 3.

<20> The composition according to any of the clauses <1> to <19> characterized in that the total concentration of one or more compound(s) according to group b) is in the range of 0.5% to 15% by weight, preferably in the range of 1% to 10% by weight, further more preferably in the range of 2% to 8% by weight, calculated to the total weight of the composition.

<21> The composition according to any of the clauses <1> to <20> characterized in that it comprises one or more conditioning compound(s) as compound(s) according to group c), preferably selected from silicones, fatty alcohols, and/or cationic surfactants, and/or their salt(s), and/or their mixtures.

<22> The composition according to clause <21> characterized in that one or more compound(s) according to group c) is/are silicones, preferably aminated silicones.

<23> The composition according to any of the clauses <21> to <22> characterized in that one or more compound(s) according to group c) is/are cationic surfactant(s), and/or their salt(s), preferably selected from quaternary ammonium surfactants and/or amidopropylamino surfactants, and/or their salt(s), and/or their mixtures.

<24> The composition according to any of the clauses <21> to <23> characterized in that the total concentration of one or more compound(s) according to group c) is in the range of 0.1% to 5% by weight, preferably in the range of 0.25% to 3% by weight, more preferably in the range of 0.5% to 2% by weight, calculated to the total weight of the composition.

<25> The composition according to any of the clauses <21> to <23> characterized in that the total concentration of silicones, preferably aminated silicones, as one or more compound(s) according to group c) is in the range of 0.1% to 5% by weight, preferably in the range of 0.25% to 3% by weight, more preferably in the range of 0.5% to 2% by weight, calculated to the total weight of the composition.

<26> The composition according to any of the clauses <21> to <23> characterized in that the total concentration of cationic surfactant(s), and/or their salt(s), preferably selected from quaternary ammonium surfactants and/or amidopropylamino surfactants, and/or their salt(s), and/or their mixtures, as one or more compound(s) according to group c) is in the range of 0.1% to 5% by weight, preferably in the range of 0.25% to 3% by weight, more preferably in the range of 0.5% to 2% by weight, calculated to the total weight of the composition.

<27> The composition according to any of the clauses <1> to <26> characterized in that the composition comprises less than 1% by weight of reducing agent(s), preferably less than 0.1% by weight of reducing agent(s), calculated to the total weight of the composition

<28> The composition according to any of the clauses <1> to <27> characterized in that the composition is free of reducing agents.

<29> The composition according to any of the clauses <1> to <28> characterized in that the composition comprises less than 1% by weight of oxidizing agent(s), preferably less than 1% by weight of oxidizing agent(s), calculated to the total weight of the composition.

<30> The composition according to any of the clauses <1> to <29> characterized in that the composition is free of oxidizing agents.

<31> The composition according to any of the clauses <1> to <30> characterized in that it is a temporary straightening composition for keratin fibers, preferably a temporary straightening composition for keratin fibers with heat at 50° C. or above, more preferably with heat at 120° C. or above.

<32> The composition according to any of the clauses <1> to <31> characterized in that it is a temporary straightening composition.

<33> The composition according to any of the clauses <1> to <32> characterized in that it comprises water at 50% by weight or more, preferably to have a water content of 60% by weight or more, more preferably to have a water content of 70% by weight or more, calculated to the total weight of the composition.

<34> The composition according to any of the clauses <1> to <33> characterized in that the pH of the composition is in the range of 1.25 to 5, more preferably in the range of 1.5 to 4.5, further more preferably in the range of 2 to 4.

The disclosure of the present invention is also directed to:

<35> A two-chamber packaging device having:
a first chamber comprising a first aqueous composition comprising one or more compound(s) according to group a) as defined in any of the clauses <1> to <34>, and
a second chamber comprising a second aqueous composition comprising one or more compound(s) according to group b) as defined in any of the clauses <1> to <34>.

<36> The packaging according to clause <35> characterized in that the first and second chamber compositions are kept separate until directly prior to use onto keratin fibers.

<37> The packaging according to any of the clauses <35> to <36> characterized in that the mixture of the compositions has a pH in the range of 1 to 6, preferably in the range of 1.25 to 5, more preferably in the range of 1.5 to 4.5, further more preferably in the range of 2 to 4.

<38> The packaging according to any of the clauses <35> to <37> characterized in that the first and second chamber compositions have a pH in the range of 1 to 6, preferably in the range of 1.25 to 5, more preferably in the range of 1.5 to 4.5, further more preferably in the range of 2 to 4.

<39> The packaging according to any of the clauses <35> to <37> characterized in that the first chamber composition has a pH in the range of 1 to 6, preferably in the range of 1.25 to 5, more preferably in the range of 1.5 to 4.5, further more preferably in the range of 2 to 4, and the second chamber composition has a pH in the range of 7 to 12.

<40> The packaging according to any of the clauses <35> to <37> characterized in that the first chamber composition has a pH in the range of 7 to 12, and the second chamber composition has a pH in the range of 1 to 6, preferably in the range of 1.25 to 5, more preferably in the range of 1.5 to 4.5, further more preferably in the range of 2 to 4.

<41> The packaging according to any of the clauses <35> to <40> characterized in that the packaging comprises a valve configured to subsequently or simultaneously dispense from two chambers upon actuation.

<42> The packaging according to clause <41> characterized in that the packaging comprises a valve configured to simultaneously dispense from two chambers upon actuation.

The disclosure of the present invention is also directed to:

<43> A kit-of-parts having a first component with an aqueous composition comprising one or more compound(s) according to group a) as defined in any of the clauses <1> to <34>, and a second component with an aqueous composition comprising one or more compound(s) according to group b) as defined in any of the clauses <1> to <34>.

<44> The kit-of-parts according to clause <43> characterized in that the first and second components are kept separate until directly prior to use onto keratin fibers.

<45> The kit-of-parts according to any of the clauses <43> to <44> characterized in that the mixture of the components has a pH in the range of 1 to 6, preferably in the range of 1.25 to 5, more preferably in the range of 1.5 to 4.5, further more preferably in the range of 2 to 4.

<46> The kit-of-parts according to any of the clauses <43> to <45> characterized in that the first and second components of the kit have a pH in the range of 1 to 6, preferably in the range of 1.25 to 5, more preferably in the range of 1.5 to 4.5, further more preferably in the range of 2 to 4.

<47> The kit-of-parts according to any of the clauses <43> to <45> characterized in that the first component of the kit has a pH in the range of 1 to 6, preferably in the range of 1.25 to 5, more preferably in the range of 1.5 to 4.5, further more preferably in the range of 2 to 4, and the second component of the kit has a pH in the range of 7 to 12.

<48> The kit-of-parts according to any of the clauses <43> to <45> characterized in that the first component of the kit has a pH in the range of 7 to 12, and the second component has a pH in the range of 1 to 6, preferably in the range of 1.25 to 5, more preferably in the range of 1.5 to 4.5, further more preferably in the range of 2 to 4.

<49> The kit-of-parts according to clause <48> characterized in that the first component and/or the second component further comprise one or more compound(s) according to group c) as defined in any of the clauses <21> to <26>.

<50> The kit-of-parts according to any of the clauses <43> to <49> characterized in that the first and second component comprise less than 1% by weight of reducing agent(s), preferably less than 0.1% by weight of reducing agent(s), calculated to the total weight of the composition, more preferably they are free of reducing agents.

<51> The kit-of-parts according to any of the clauses <43> to <50> characterized in that the first and second component comprise less than 1% by weight of oxidizing agent(s), preferably less than 1% by weight of oxidizing agent(s), calculated to the total weight of the composition, more preferably they are free of oxidizing agents.

The disclosure of the present invention is also directed to:

<52> A straightening process for keratin fibers comprising the steps in the following order of:
i) providing the composition as defined in any of the clauses <1> to <34>, or providing and mixing the first and second component as defined in any of the clauses <43> to <51>, or providing and dispensing and optionally mixing the compositions as defined in any of the clauses <35> to <42>, yielding a composition having a pH in the range of 1 to 6, preferably in the range of 1.25 to 5, more preferably in the range of 1.5 to 4.5, further more preferably in the range of 2 to 4,
ii) applying the composition of step i) onto keratin fibers,
iii) optionally covering the keratin fibers with a moisture barrier,
iv) leaving the composition onto the keratin fibers for a time period of 1 min to 24 h, preferably for a time period of 1 min to 120 min,
v) optionally rinsing-off the hair,
vi) drying the hair,
vii) applying heat to the keratin fibers at a temperature in the range of 50° C. to 230° C.,
viii) rinsing-off the keratin fibers and optionally drying the keratin fibers.

<53> The process according to clause <52> characterized in that the temperature of step vii) is in the range of 90° C. to 230° C., preferably in the range of 120° C. to 230° C., more preferably in the range of 150° C. to 230° C.

<54> The process according to any of the clauses <52> to <53> characterized in that it is a process for temporary straightening keratin fibers, preferably for temporary straightening keratin fibers.

<55> The process according to any of the clauses <52> to <54> characterized in that the time period of step iv) is in the range of 2 min to 60 min, preferably in the range of 5 min to 45 min, more preferably in the range of 10 min to 40 min.

<56> The process according to any of the clauses <52> to <55> characterized in that heat in step vii) is applied with a straightening iron.

The following examples are to illustrate the invention, but not to limit it.

### Examples

TABLE 1
The following compositions were prepared by subsequently
dissolving the compounds in water.

| | Inv. | Inv. | Inv. | Inv. | Comp. | Comp. | Comp. | Comp. | Comp. |
|---|---|---|---|---|---|---|---|---|---|
| | ex. 1 | ex. 2 | ex. 3 | ex. 4 | ex. 1 | ex. 2 | ex. 3 | ex. 4 | ex. 5 |
| Ingredient | [% by weight] | | | | | | | | |
| Citric acid | 1.0 | 5.0 | 7.0 | 10.0 | 1.0 | 5.0 | 7.0 | 10.0 | — |
| Glycerol | 0.4 | 2.2 | 3.1 | 4.4 | — | — | — | — | 2.2 |
| NaOH | pH ad 3.5 | | | | | | | | |
| Water | Ad 100.0 | | | | | | | | |
| Hair volume | 0.83 | 0.58 | 0.59 | 0.84 | 1.00 | 0.85 | 0.97 | 0.88 | 1.07 |
| after 10 washes | | | | | | | | | |
| Volume | 83% | 68% | 61% | 95% | — | — | — | — | — |
| Reduction [%] | | | | | | | | | |

Hair volume after 10 washes is normalized to the untreated hairstreak.

### Methods

Indian Frizzy hair (Fischbach und Miller, Laupheim, Germany) was washed with a shampoo available under the tradename Dualsenses Deep Cleansing Shampoo, combed 5-times from each side and allowed to air-dry in a controlled environment at 22° C. and 60% relative humidity. The initial volume of the hair strand was determined using an optical image analyzing software as detailed below. The strand was then treated with the compositions above by dipping the strand into the compositions. After 30 seconds, the strand was removed, put on a glass, and covered with a plastic foil to avoid evaporation. The product was allowed to act for 20 minutes at room temperature, excess product was removed and the strand was blow dried for 4 minutes. The dry strand was then treated 6-times with a straightening iron at 200° C. and the volume of the hair strand was determined using an optical image analyzing software as detailed below.

### Durability Test:

The test strand was washed with the shampoo from above by wetting the hair with warm water for 10 seconds, applying the shampoo for 30 seconds by hand, rinsing the hair with warm water for 30 seconds, combing each side 5-times and then rinse again for 30 seconds. The dripping wet strand was allowed to air dry in a controlled environment at 22° C. and 60% relative humidity. On the next day the hair volume was measured using an optical image analyzing software. This process was repeated until 10 washes were achieved.

### Analysis of Hair by Optical Image Analyzation:

The hair strand was put into a white photobox with fixed positions, distances and light settings. A greyscale digital photo was taken, then the photograph was converted into a black and white image using ImageJ software. The black pixels in the fixed frame were counted. A high number of black pixels correlated to a higher volume of the hair streak.

1. An aqueous straightening composition for keratin fibers, having a pH in the range of 1 to 6, and comprising:
a) one or more tridentate carboxylic acid, and/or their salt(s), and/or their hydrates, and/or their mixtures, and
b) one or more C₂-C₄ trialcohol(s), and/or their mixtures,
wherein the total concentration of compound(s) according to group a) is 1% by weight or more, calculated to a total weight of the composition.

2. (canceled)

3. The aqueous straightening composition according to claim 1, wherein the one or more compound(s) according to group a) is/are a citric acid, isocitric acid, aconitic acid, propane-1,2,3-tricarboxylic acid, trimesic acid, and/or their salt(s), and/or their hydrates, and/or their mixtures.

4. The aqueous straightening composition according to claim 1, wherein the total concentration of compound(s) according to group a) is in the range of 1% to 10% by weight, calculated to the total weight of the composition.

5. The aqueous straightening composition according to claim 1, wherein the one or more compound(s) according to group b) is glycerol.

6. The aqueous straightening composition according to claim 1, wherein the weight ratio of compound(s) according to group a) to compound(s) according to group b) is in the range of 0.2 to 5.

7. The aqueous straightening composition according to claim 1, wherein the total concentration of the one or more compound(s) according to group b) is in the range of 0.5% to 10% by weight, calculated to the total weight of the composition.

8. The aqueous straightening composition according to claim 1, further comprising one or more conditioning compound(s) as compound(s) according to group c) selected from silicones, fatty alcohols, and/or cationic surfactants, and/or their salt(s), and/or their mixtures.

9. The aqueous straightening composition according to claim 1, wherein the pH of the composition is in the range of 2 to 4.

10. The aqueous straightening composition according to claim 1, further comprising less than 1% by weight of reducing agent(s), calculated to the total weight of the composition.

11. The aqueous straightening composition according to claim 1, further comprising less than 1% by weight of oxidizing agent(s), calculated to the total weight of the composition.

12. A two-chamber packaging device having:
a first chamber comprising a first aqueous composition comprising one or more compound(s) according to group a) as defined in claim 1, and
a second chamber comprising a second aqueous composition comprising one or more compound(s) according to group b) as defined in claim 1.

13. A kit-of-parts having a first component with an aqueous composition comprising one or more compound(s) according to group a) as defined in claim 1, and a second component with an aqueous composition comprising one or more compound(s) according to group b) as defined in claim 1.

14. A straightening process for keratin fibers comprising the steps in the following order of:
i) providing the aqueous straightening composition as defined in claim 1, or providing and mixing the first and second component as defined in claim 13, or providing and dispensing and optionally mixing the compositions as defined in claim 12, yielding a composition having a pH in the range of 1 to 6,
ii) applying the composition of step i) onto keratin fibers,
iii) optionally covering the keratin fibers with a moisture barrier,
iv) leaving the composition onto the keratin fibers for a time period of 1 min to 24 h,
v) optionally rinsing-off the hair,
vi) drying the hair,
vii) applying heat to the keratin fibers at a temperature in the range of 50° C. to 230° C.,
viii) rinsing-off the keratin fibers and optionally drying the keratin fibers.

15. The process according to claim 14, wherein the temperature of step vii) is in the range of 150° C. to 230° C.

16. The process according to claim 14, wherein the heat in step vii) is applied with a straightening iron.

17. The process according to claim 14, wherein the time period of step iv) is in the range of 1 min to 120 min.

18. The aqueous straightening composition according to claim 1, wherein the one or more compound(s) according to group a) is/are selected from citric acid or isocitric acid, and/or their salt(s), and/or their hydrates, and/or their mixtures.

19. The aqueous straightening composition according to claim 1, wherein the total concentration of compound(s) according to group a) is in the range of 3% to 8% by weight, calculated to the total weight of the composition.

20. The aqueous straightening composition of claim 1, wherein the weight ratio of compound(s) according to group a) to compound(s) according to group b) is in the range of 1 to 3.

21. The aqueous straightening composition of claim 1, wherein the total concentration of the one or more compound(s) according to group b) is in the range of 2% to 8% by weight, calculated to the total weight of the composition.
